(19) United States
(12) Patent Application Publication
(10) Pub. No.: US 2016/0025738 A1
(43) Pub. Date: Jan. 28, 2016

(54) PROGNOSTIC MARKER TO DETERMINE THE RISK FOR EARLY-ONSET PREECLAMPSIA

(71) Applicant: IQ PRODUCTS B.V., DL Groningen (NL)

(72) Inventor: Joost Henric Nicolaas Schuitemaker, Tolbert (NL)

(21) Appl. No.: 14/772,893

(22) PCT Filed: Mar. 3, 2014
(86) PCT No.: PCT/EP2014/054053

§ 371 Date: Sep. 4, 2015

Related U.S. Application Data
(60) Provisional application No. 61/772,501, filed on Mar. 4, 2013.

(30) Foreign Application Priority Data
Jun. 10, 2013 (EP) 13171331.5

(51) Int. Cl. G01N 33/68; G01N 21/49; G01N 27/62

(52) U.S. Cl. CPC G01N 33/689; G01N 27/62; G01N 21/49; G01N 2800/368

(57) ABSTRACT

The present application relates to an in vitro method for identifying a pregnancy related syndrome selected from the group consisting of pre-eclampsia, eclampsia, Hemolysis Elevated Liver enzymes and Low Platelets (HELLP) and intra-uterine growth restriction (IUGR), the method including (i) measuring the amount of ESM-1 in a biological fluid sample from a pregnant subject, wherein the pregnant subject is between week 1 to 20 of gestation; (ii) comparing said amount of ESM-1 to a reference value, and (iii) identifying the subject as being likely to have or develop the pregnancy related syndrome based on a comparison of the amount of ESM-1 to the reference value. A device and a kit for identifying such a pregnancy related syndrome are also claimed.

### FIELD OF THE INVENTION

The invention relates to the prediction, especially in an early stage of gestation, and diagnosis, in the later stages of gestation, of preeclampsia.

### BACKGROUND OF THE INVENTION

Methods for diagnosing preeclampsia are known in the art. WO2013071703 for instance describes a means for rapidly detecting Adipsin for diagnosing preeclampsia comprises a water-adsorbing pad, a nitrocellulose membrane, a gold labeling pad and a sampling pad, all of which are successively joined from the top down and fixed in the base plate. The gold-labeling pad is partly overlapped by the sampling pad. A detecting line coated by rabbit anti-human Adipsin polyclonal antibody, or goat anti-human Adipsin polyclonal antibody or mouse anti-human Adipsin polyclonal antibody and a controlling line coated by goat anti-mouse IgG polyclonal antibody are provided on the nitrocellulose membrane. The detecting line is located downstream and spaced from the controlling line. The gold-labeling pad is made of water adsorbing material, and coated by gold labeled mouse anti-human Adispin monoclonal antibody. A test kit for rapidly detecting Adipsin for diagnosing preeclampsia comprises an outer house and the said means therein. The method for preparing the said detecting means comprise: 1) coating of the detecting line and the controlling line; 2) preparing the gold labeling pad; 3) combination.

WO2014001244 describes a method for diagnosing whether a pregnant subject is not at risk for Preeclampsia within a short window of time comprising a) determining the amount of at least one angiogenesis biomarker selected from the group consisting of sFlt-1, Endoglin and P1GF in a sample of said subject, and b) comparing the amount with a reference, whereby a subject being not at risk for developing preeclampsia within a short period of time is diagnosed if the amount is identical or decreased compared to the reference in the cases of sFlt-1 and Endoglin and identical or increased in the case of P1GF, wherein said reference allows for making the diagnosis with a negative predictive value of at least about 98%. US20050170444 describes methods for diagnosing Preeclampsia and Eclampsia or a propensity to develop Preeclampsia or Eclampsia by detecting the levels of placental growth factor in a subject.

### SUMMARY OF THE INVENTION

Preeclampsia is a syndrome which is characterized by high blood pressure (>140/90) and significant amounts of protein in the urine (>300 mg/24 hrs urine) predominantly during the last weeks of pregnancy. Two forms are clinically recognized; the early-onset preeclampsia, which appears before the 34th week gestational age (GA), and the late-onset preeclampsia, which appears after the 34th week of GA. Untreated the condition might result in, for mother and child, long term effects and even a life-threatening situation. And although some women benefit from a low dose Aspirin supplementation or can be stabilized by intravenous magnesium sulfate, the best treatments for Eclampsia or advancing preeclampsia are abortion or delivery. This makes monitoring for and treatment of the early-onset preeclampsia even more important since this increases the chances for mother and child, simply because of the fact that delivery should be delayed as long as possible. A lot of research is done to find the mechanism that results to this syndrome. In this research several factors are found that might play a role in the etiology of the disease. The maternal immune system and the fact whether gestational immune tolerance is build effectively seems to play an important part. The current understanding of the syndrome is that it is a two-stage process. The initial lack of gestational immune tolerance of the placental cytotrophoblasts may lead to inadequately remodeled spiral arteries and a shallow implantation, which in turn lead to downstream hypoxia. This hypoxia of the placenta seems to be an important factor, since it is followed by the release of soluble factors in the maternal circulation. Some soluble factors, like soluble Fms-like tyrosine kinase-1 (sFlt-1 or sVEGFR-1) and soluble Endoglin (sEng), have been described as markers which are differentially expressed in preeclamptic pregnancies after week 20 of gestation compared to healthy pregnancies. These factors currently are used for screening during pregnancy and concentrations give indications of the severity of the disease.

A disadvantage of present solutions to detect Preeclampsia or Eclampsia and other pregnancy related syndromes such as Hemolysis Elevated Liver enzymes and Low Platelets (HELLP), and Intra Uterine Growth Restriction (IUGR) is that those syndromes are detectable late in the pregnancy. This prevent taking measure early during pregnancy to prevent the occurrence or reduce the severity of the syndrome. Knowing early during pregnancy that a pregnant woman will develop such syndrome also reduced unnecessary preventive measures, such as e.g. the standard use of aspirin. The subject herein is especially a woman.

In 1996 a new marker for endothelial cells was discovered by Lassalle et al. (J. Biol. Chem. 1996, 271:20458-20464). This proteoglycan of 50 kD was called endothelial cell-specific molecule 1 (ESM-1) and is also known as Endocan. The expression and secretion of ESM-1 is upregulated by pro-inflammatory molecules, such as TNF alpha, and in the presence of pro-angiogenic factors such as VEGF. The molecule has been described to be upregulated in inflammatory conditions like sepsis, but also in cancer.

As indicated above, ESM-1 plays a role in the regulation of angiogenesis and is released by activated endothelial cells. It was tested whether ESM-1 is increased in preeclampsia (PE). Plasma samples from high risk pregnancies divided in 23 healthy (CON), 11 severe early-onset PE (SE) and 7 severe late-onset PE (SL) pregnancies were collected at regular intervals between week 8 and birth. ESM-1 was measured by ELISA. Between GA week 24 and birth, ESM-1 concentrations were significantly increased in both early and late preeclampsia compared to controls (Mann Whitney, p<0.05). Surprisingly, the concentration of ESM-1 also differed between the three groups at weeks 12 and 16. The ESM-1 concentration of healthy pregnancies (mean±SEM:1857±861 pg/ml) and those that developed severe late-onset PE (1298±371 pg/ml) are comparable, but in those pregnancies that develop severe early-onset PE, the concentration (410±355 pg/ml) is significantly lower as compared with healthy pregnancies. Hence, ESM-1 concentrations are increased during early and late severe preeclampsia, which may be due to endothelial cell activation in these conditions. Surprisingly, since ESM-1 is decreased at 12-16 weeks in patients that later on develop early onset severe PE, it might be a prognostic marker which can determine the risk of women to develop severe early-onset PE already as early as 1-20 weeks, such as 8-20 weeks, especially 8-16 weeks, even more especially 12 to 16 weeks of gestation.

Hence, in a first aspect, the invention provides a method for diagnosing whether a pregnant subject has, or is having a predisposition for the development of, preeclampsia, eclampsia, HELLP or IUGR, or not, said method comprising the determination of the amount of ESM-1 in a sample of said subject. Especially, the invention provides in a first aspect an (in vitro) method for identifying from a biological sample from a pregnant subject a pregnancy related syndrome selected from the group consisting of Preeclampsia, Eclampsia, Hemolysis Elevated Liver enzymes and Low Platelets (HELLP), and Intra Uterine Growth Restriction (IUGR), the method including (i) (using an assay specific to ESM-1 and) measuring the amount of ESM-1 in the biological sample, wherein the biological sample (especially a biological fluid sample) is from the pregnant subject being in any one week of gestation between week 1 to 20 of gestation, such as week 8-20 (or earlier), especially week 8-16, even more especially week 12-16; and (ii) comparing said amount of ESM-1 to a reference value. The method, as indicated above, may especially further comprise (iii) identifying the subject as being likely to have or develop the pregnancy related syndrome based on a comparison of the amount of ESM-1 to the reference value. With the present invention, already before week 24, even before week 20, the likeliness of developing such syndrome or even the presence may be detected. Hence, in a very early stage, a treatment or prevention therapy may be started. Herein, the phrase “measuring the amount of ESM-1” may also relate to measuring an equivalent of ESM-1 or measuring a metabolite of ESM-1. In an embodiment said ESM-1 is the level of free ESM-1, bound ESM-1, a metabolite of ESM-1 or total ESM-1. Especially, said amount of ESM-1 is the level of free ESM-1 in the biological sample. The reference value may especially be a reference value measured in a sample from a woman having a healthy pregnancy. Especially, a healthy pregnancy relates to a pregnancy of the subject that does not have or develops any of the herein indicated pregnancy related syndromes (during the entire pregnancy). This may especially indicate that the reference value is such ESM-1 value of a sample of a pregnant subject, which later appeared not to develop or have such pregnancy related syndrome. Such reference value may especially be a mean value from a group of pregnant subjects that appeared all not to have or develop such pregnancy related syndrome. As indicated above (and also below), the striking observation was done hat those pregnant subjects that had already the pregnancy related syndrome or later appeared to develop such syndrome had substantial lower ESM-1 values in biological samples of these subjects in taken in week 20 (after gestation) or earlier. Hence, in a specific embodiment the method further comprises (iii) identifying the subject as being likely to have or develop the pregnancy related syndrome based on a comparison of the amount of ESM-1 to the reference value when said amount of ESM-1 is smaller than the ESM-1 reference value. Hence, especially the reference value is the amount of ESM-1 in biological samples of pregnant subjects having a healthy pregnancy. Especially, the reference values may include a single reference value for a specific gestation period or a plurality of reference values for a plurality of periods within the gestation period. Especially, the measured ESM-1 value is compared to a reference value indicative for the time of gestation at which the biological fluid sample of the pregnant subject is taken. For instance, there may be specific reference values for week 8-12 and reference values for week 12-16, etc. Optionally, there may be different reference values for different types of samples. The reference value may be a number but may also be a color, a color intensity or lack thereof, be an amount of radioactivity, or an amount of light or lack thereof, as is known in the art.

Especially, the sample is a fluid sample, i.e. a biological fluid sample. In an embodiment, said sample is a blood, plasma, serum, urine or saliva from the subject. Especially, said biological sample comprises a blood sample, a plasma sample, or a serum sample from said pregnant subject. However, the sample may also include cerebrospinal fluid (CSF) or other biological fluid. Optionally, the biological sample may include a placenta biopt. Even more especially, said biological sample is from a pregnant subject being in any one week of gestation between weeks 12 to 16 of gestation. In yet even a more specific embodiment, said biological sample comprises a plasma example and the pregnant subject is evaluated to have the pregnancy related syndrome when the ESM-1 value is in the range of 410±355 pg/ml.

In a specific embodiment, the amount of ESM-1 of the biological sample of the pregnant subject is indicative of the pregnancy related syndrome when the amount is equal to or less than 80%, especially equal to or less than 70%, especially equal to or less than 60%, such as equal to or less than 50%, like even more especially equal to or less than 40%, of the amount of ESM-1 of biological samples of pregnant subjects having a healthy pregnancy. Of course, comparisons are made between samples taken in the same period of pregnancy, such as in the gestation period of in weeks 1-20, like weeks 12-16. Hence, the amount of ESM-1 of the biological sample of the pregnant subject may be indicative of the pregnancy related syndrome when the amount is equal to or less than 80%, especially equal to or less than 70%, especially equal to or less than 60%, such as equal to or less than 50%, like even more especially equal to or less than 40%, of the amount of ESM-1 of biological samples of pregnant subjects having a healthy pregnancy (wherein the biological sample of the pregnant subject and the reference value relate to samples in the same week (especially selected from week 1-2) of gestation.

Especially, the assay is an antibody based assay, i.e. the assay uses an antibody to detect the ESM-1 protein (or equivalent or metabolite). In a specific embodiment, the assay comprises an ELISA (Enzyme-linked immunosorbent assay) assay, a turbidimetric assay, radioimmunosorbent assay (RIST), a radioimmunoassay (RIA), a direct or indirect immunofluorescence assay, a particle gel immuno assay (PaGIA), or a lateral flow assay. Even more especially, the assay comprises an ELISA. Alternatively or additionally, the assay comprises a lateral flow assay. Alternatively or additionally, the assay comprises an enzymatic assay, a colorimetric assay, or a luminescent assay. The measurements may alternatively or additionally also include mass spectrometry measurements, especially of metabolites of ESM-1. Hence, in an embodiment measuring the amount of ESM-1 in the biological sample includes using mass spectrometry (MS).

In yet a further embodiment, not the ESM-1 is directly measured but RNA coding for ESM-1 is detected, i.e. especially the amount of RNA coding for ESM-1 is detected. Hence, in a specific embodiment measuring the amount of ESM-1 in the biological sample comprises a method to do a quantification of RNA coding for ESM-1 in cells or cell-free RNA in the biological sample, especially a fluid (i.e. a liquid). In yet a further embodiment, the invention involves a method wherein measuring the amount of ESM-1 in the biological sample comprises a method to quantify the amount of protein via the amount of RNA coding for ESM-1. For instance, in an embodiment said quantification is done via RT-PCR, PCR, hybridization or other means to determine the amount of nucleotides. In addition to the ESM-1 marker, also one or more other markers may be used. The absolute values thereof may be compared to absolute reference values. However, alternatively or additionally, a ratio between the ESM-1 marker and the other marker may be used to be compared with a reference ratio. For instance, in a specific embodiment, the method may further comprise (iv) using (selecting) a secondary marker selected from the group consisting of soluble Fms-like tyrosine kinase-1 (sFlt1), Vascular Endothelial Growth Factor (VEGF), Placental Growth Factor (P1GF), Hepatocyte Growth Factor (HGF), soluble Endoglin, placental protein 13 (pp-13), Pregnancy-associated Plasma Protein A (PAPP-A) and Growth Differentiation Factor 15 (GDF-15), (v) using an assay specific for the secondary marker and measuring the amount of the secondary marker in the biological sample, and optionally comparing said amount of said secondary marker to a secondary marker reference value. This may further enhance certainty of the identification process. Alternative or additional to the above indicated secondary marker, also one or more or pikachurin and a hemopexin may be chosen. Pikachurin, also known as agrin-like protein (AGRINL) and EGF-like, fibronectin type-III and laminin G-like domain-containing protein (EGFLAM), is a protein that in humans is encoded by the EGFLAM gene. Hemopexin (or haemopexin or HPX), also known as beta-1B-glycoprotein is a protein that in humans is encoded by the HPX gene and belongs to hemopexin family of proteins.

In a further specific embodiment, the method may further comprise identifying the subject as being likely to have or develop the pregnancy related syndrome based on a comparison of the amount of ESM-1 to a reference value, and based on a comparison of the amount of said one or more secondary markers with the corresponding secondary marker reference value. Alternatively or additionally, in a specific embodiment, the method may further comprise determining a ratio of the amount of ESM-1 to the amount of the secondary marker, selecting a ratio reference value, and comparing the ratio with the ratio reference value. Hence, in yet a further specific embodiment, the method may further comprise identifying the subject as being likely to have or develop the pregnancy related syndrome based on a comparison of the amount of ESM-1 to a reference value, and based on a comparison of the ratio of the amount of ESM-1 to the amount of the secondary marker to the ratio reference value.

Alternatively or additionally, the method may include a multi-stage process, wherein samples taken at different times, like before week 12 and after week 16, or before week 20, and after week 20, etc., may be taken and may be used to identify whether or not the pregnant subject has is will likely develop the pregnancy related syndrome. Hence, in a specific embodiment, the method may (further) comprise providing a first biological sample from a subject extracted on a first occasion, and providing a second biological sample from the subject extracted on a second occasion, using an assay specific to ESM-1, measuring the amount of ESM-1 in the first biological sample, constituting a first amount, using an assay specific to ESM-1, measuring the amount of ESM-1 in the second biological sample, and constituting a second amount. The second occasion is especially later in time than the first occasion. At least the first sample is especially taken in week 20 of gestation or earlier (like 12-16). Further, the method and use, as well as the application of the kit may include extracting one biological sample, two biological samples, but also more than two biological samples. Especially, these are taken at different times, such as with one or more days in between, or one or more weeks in between. Especially in a specific embodiment, the method may further comprise identifying the subject as being likely to have or develop the pregnancy related syndrome based on a comparison of the first amount and the second amount.

In a specific embodiment, the method may further comprise the step of uterine artery Doppler screening. This screening may advantageously give additional information about the placentation. Poor placentation is known to be an additional indication for the risk of the development of pregnancy related problems.

In yet a further aspect, the invention also provides a device for identifying a pregnant subject that is likely to have or develop a pregnancy related syndrome selected from the group consisting of Preeclampsia, Eclampsia, HELLP and IUGR, said device comprising: (a) an analyzing unit comprising a detection agent for ESM-1, for instance being an antibody, a (recombinant) receptor for ESM-1 or an aptamer, which allows the determination of the amount of ESM-1; (b) an evaluation unit comprising a data processor having implemented necessary algorithms for comparing the amount of ESM-1 determined with a reference value stored in a database in order to determine whether a pregnant subject is likely to have or develop preeclampsia, eclampsia, HELLP and/or IUGR. Especially, in an embodiment the device may further comprise an analyzing unit comprising a detection agent for sFlt1, VEGF, P1GF, HGF, sEndoglin, pp-13, PAPP-A or GDF-15, which allows the determination of the amount of sFlt1, VEGF, P1GF, HGF, sEndoglin, pp-13, PAPP-A or GDF-15, and an evaluation unit comprising a data processor having implemented necessary algorithms for comparing the amount of sFlt1, VEGF, P1GF, HGF, sEndoglin, pp-13, PAPP-A or GDF-15 determined with reference values stored in a database and calculate the ratio between the measured markers in order to determine whether a pregnant subject is likely to have or develop preeclampsia, eclampsia, HELLP and/or IUGR. As will be clear to a person skilled in the art, the analyzing units and the evaluation units, ESM-1 and one ore more of the other markers, may optionally be a single analyzing unit and/or a single evaluation unit, respectively. Especially, in yet a further embodiment the evaluation unit is also capable of giving therapeutic recommendations. Alternative or additional to the above indicated secondary marker, also one or more or pikachurin and a hemopexin may be chosen.

In yet a further aspect, the invention also provides the use of an evaluation of the amount of ESM-1 in an extracted biological sample from a pregnant subject for identifying whether the pregnant subject is likely to have or develop a pregnancy related syndrome selected from the group consisting of Preeclampsia, Eclampsia, HELLP and IUGR. Even more especially, the invention provides in an embodiment the in vitro use of an extracted biological sample from a pregnant subject for identifying whether the pregnant subject is likely to have or develop a pregnancy related syndrome selected from the group consisting of Preeclampsia, Eclampsia, HELLP and IUG, the use comprising: (a) using an assay specific to ESM-1, measuring the amount of ESM-1 in the biological sample; (b) comparing said amount of ESM-1 to a reference value; and (c) identifying the subject as being likely to have or develop the pregnancy related syndrome.

In yet a further aspect, the invention provides a kit for identifying whether a pregnant subject is likely to have or develop a pregnancy related syndrome selected from the group consisting of Preeclampsia, Eclampsia, HELLP and IUG, said kit comprising: (a) an analyzing unit comprising a detection agent for ESM-1; (b) an evaluation unit for determining whether a pregnant subject is likely to have or develop the pregnancy related syndrome, based on a result provided by the analyzing unit. Especially, the invention provides an embodiment of the kit further comprising a manual or a reference to a manual, such as a remote manual (like a database on a server). Yet even more especially, the invention provides an embodiment of the kit, wherein the manual includes instructions how to extract biological sample from a pregnant subject and/or how to use the analyzing unit and/or how to use the evaluation unit.

In an embodiment, the evaluation unit comprises a color scheme, wherein the analyzing unit is configured to provide a color reaction wherein the color is dependent upon the amount of ESM-1 in an extracted biological sample from a pregnant subject. Especially, the invention provides an embodiment of the kit comprising: (b) an evaluation unit comprising a data processor for determining whether a pregnant subject is likely to have or develop preeclampsia, eclampsia, HELLP and/or IUGR. Especially, the invention provides an embodiment of the kit comprising: (b) an evaluation unit comprising a data processor having implemented necessary algorithms for comparing the amount ESM-1 determined with reference values stored in a database in order to determine whether a pregnant subject is likely to have or develop the pregnancy related syndrome. In a specific embodiment of the kit, said kit comprises: (a) an analyzing unit comprising a detection agent for ESM-1 and one or more of sFlt1, VEGF, P1GF, HGF, sEndoglin, pp-13, PAPP-A and GDF-15. Hence, the invention may also provide an embodiment of the kit comprising and analyzing unit comprising a detection agent for ESM-1, and a detection agent for one or more of sFlt1, VEGF, P1GF, HGF, sEndoglin, pp-13, PAPP-A and GDF-15, which allows the determination of the amount of ESM-1, and one or more of sFlt1, VEGF, P1GF, HGF, sEndoglin, pp-13, PAPP-A and GDF-15. Yet even more especially, the invention provides an embodiment of the kit comprising an evaluation unit comprising a data processor having implemented necessary algorithms for comparing the amount ESM-1, and one or more of sFlt1, VEGF, P1GF, HGF, sEndoglin, pp-13, PAPP-A and GDF-15 determined with reference values stored in a database and calculate the ratio between the measured markers in order to determine whether a pregnant subject is likely to have or develop the pregnancy related syndrome. Alternative or additional to the above indicated secondary marker, also one or more or pikachurin and a hemopexin may be chosen. As indicated above, in an embodiment the analyzing unit may comprise an assay. Even more especially, in an embodiment the analyzing unit comprises an ELISA (Enzyme-linked immunosorbent assay) assay, a turbidimetric assay, radioimmunosorbent assay (RIST), radioimmunoassay (RIA), direct or indirect immunofluorescence, particle gel immuno assay (PaGIA), or a lateral flow assay.

In an embodiment (of the kit), the manual includes information to extract biological sample from a pregnant subject in any one week of gestation, especially between week 1 to 20 of gestation, even more especially between weeks 12 to 16.

Alternatively or additionally the invention provides an embodiment of the kit wherein the analyzing unit comprises a method to do a quantification of the RNA coding for ESM-1 in cells or cell-free RNA in the biological sample, especially a biological fluid, such as a body liquid.

Hence, the method, kit or device of the invention may be used for predicting a pregnant subject in any one of week 1 to 20 of gestation to develop after week 20 early onset severe preeclampsia, based on a measurement of the amount of ESM-1 in a biological fluid sample from the pregnant subject, wherein the biological fluid sample especially comprises a blood sample, a plasma sample, or a serum sample from said pregnant subject. This will especially be the case when the ESM-1 amount is substantially smaller than the ESM-1 amount in a sample (from a pregnant subject at the same time, especially in about the same week of gestation) of a healthy pregnant subject.

As used herein the term “ESM-1” or “endocan” has its general meaning in the art and refers to the endothelial cell specific molecule-1 that is a 50-kDa dermatan sulfate proteoglycan expressed by endothelial cells in lung and kidney, among other cell types, and can be detected in human blood. As used herein the term “blood sample” refers to a whole blood, serum, or plasma sample. Typically the blood sample is drawn/collected from a patient by a physician or nurse and processed in the laboratory of the hospital. The above indicated assays may also be used in peripheral hospitals or practices and could even be in general use by midwives. Once the blood sample from the patient is prepared, the concentration of ESM-1 may be measured by any known method in the art. It is for instance referred to WO2012098219, which is herein incorporated by reference. For example, the concentration of ESM-1 may be measured by using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radio immunoassays; Immuno electrophoresis; immune precipitation, high performance liquid chromatography (FIPLC), size exclusion chromatography, solid-phase affinity, etc. As also described in WO2012098219, such methods may comprise contacting the blood sample with a binding partner capable of selectively interacting with ESM-1 present in the blood sample. The binding partner may be generally an antibody that may be polyclonal or monoclonal, preferably monoclonal. Polyclonal antibodies directed against ESM-1 can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against ESM-1 can be prepared and isolated using any technique that provides for the production of antibody molecules by continuous cell lines in culture. Techniques for production and isolation include but are not limited to the hybridoma technique originally described WO2012098219 and references cited therein. Antibodies useful in practicing the present invention also include anti-ESM-1 fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv (single chain Fv) expression libraries can be constructed to allow rapid identification of fragments having the desired specificity to ESM-1. For example, phage display of antibodies may be used. In such a method, single-chain Fv (scFv) or Fab fragments are expressed on the surface of a suitable bacteriophage, e. g., M13. Briefly, spleen cells of a suitable host, e. g., mouse, that has been immunized with a protein are removed. The coding regions of the VL and VH chains are obtained from those cells that are producing the desired antibody against the protein. These coding regions are then fused to a terminus of a phage sequence. Once the phage is inserted into a suitable carrier, e. g., bacteria, the phage displays the antibody fragment. Phage display of antibodies may also be provided by combinatorial methods known to those skilled in the art. Antibody fragments displayed by a phage may then be used as part of an immunoassay. Anti-ESM-1 monoclonal antibodies are commercially available from, for instance, Lunginnov (Lille, France), see also WO2012098219.

In another embodiment, the binding partner may be an aptamer. Aptamers are a class of molecules that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by Exponential enrichment (SELEX) of a random sequence library, as described in WO2012098219 and references cited therein. The random sequence library is obtainable by combinatorial chemical synthesis of DNA. In this library, each member is a linear oligomer, eventually chemically modified, of a unique sequence. Possible modifications, uses and advantages of this class of molecules have been indicated and described in WO2012098219, and references cited therein. The binding partners of the invention such as antibodies or aptamers, may be labelled with a detectable molecule or substance, such as a fluorescent molecule, a radioactive molecule or any others labels known in the art. Labels are known in the art that generally provide (either directly or indirectly) a signal.

Early onset severe PE especially belongs to the herein described pregnancy related syndromes (such as preeclampsia). As used herein, the term “labeled”, with regard to the antibody, is intended to encompass direct labeling of the antibody or aptamer by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody or aptamer, as well as indirect labeling of the probe or antibody by reactivity with a detectable substance. An antibody or aptamer of the invention may be labeled with a radioactive molecule by any method known in the art. For example radioactive molecules include but are not limited radioactive atom for scintigraphic studies such as 1123, 1124, Inl 11, Rel 86, Rel88. As also indicated in WO2012098219, the aforementioned assays generally involve the bounding of the binding partner (i.e. Antibody or aptamer) in a solid support. Solid supports which can be used in the practice of the invention include substrates such as nitrocellulose (e. g., in membrane or micro titer well form); polyvinylchloride (e. g., sheets or micro titer wells); polystyrene latex (e.g., beads or micro titer plates); polyvinylidine fluoride; diazotized paper; nylon membranes; activated beads, magnetically responsive beads, and the like.

More particularly, an ELISA method can be used, wherein the wells of a micro titer plate are coated with a set of antibodies against ESM-1. A blood sample containing or suspected of containing ESM-1 is then added to the coated wells. After a period of incubation sufficient to allow the formation of antibody-antigen complexes, the plate(s) can be washed to remove unbound moieties and a detectably labeled secondary binding molecule added. The secondary binding molecule is allowed to react with any captured sample marker protein, the plate washed and the presence of the secondary binding molecule detected using methods well known in the art. Typically an ELISA kit is commercially available from Lunginnov (Lille, France) or other sources, such as described in WO2012098219 and references cited therein. Measuring the concentration of ESM-1 (with or without immunoassay-based methods) may also include separation of the proteins: centrifugation based on the protein's molecular weight; electrophoresis based on mass and charge; HPLC based on hydrophobicity; size exclusion chromatography based on size; and solid-phase affinity based on the protein's affinity for the particular solid-phase that is use. Once separated, ESM-1 may be identified based on the known “separation profile” e. g., retention time, for that protein and measured using standard techniques. Alternatively, the separated proteins may be detected and measured by, for example, a mass spectrometer. Hence, presence and quantification of the analyte can be shown and done by using generally accepted techniques like, for instance, the enzyme linked immuno sorbent assay (ELISA), either in sandwich or competitive set up, a lateral flow method, a magnetic or fluorescent particle (bead) based assay and western blot, etc.

In an embodiment the amount of sFlt1, VEGF, P1GF, HGF, sEndoglin, pp-13, PAPP-A or GDF-15, or a combination of these markers, is determined in a sample in addition to the amount of ESM-1 and wherein sFlt1, VEGF, P1GF, HGF, sEndoglin, pp-13, PAPP-A or GDF-15, or combination of these markers, are compared to a reference amount of ESM-1, sFlt1, VEGF, P1GF, HGF, sEndoglin, pp-13, PAPP-A or GDF-15 for diagnosing the risk of experiencing preeclampsia, eclampsia, HELLP or IUGR. In an embodiment the ratio between ESM-1 and any of said markers or a combination of said markers in a sample is used to determine the predisposition of a pregnant subject for the development of preeclampsia, eclampsia, HELLP or IUGR, or not. In an embodiment said sample is a blood, plasma, serum, urine or saliva from the subject. In an embodiment said sample is derived from a subject being in any one week of gestation between week 1 to 20 of gestation. In an embodiment said sample is derived from a subject being in any one week of gestation between week 20 to 36 of gestation. In an embodiment said measuring levels is done on two or more occasions and a change in said levels is a diagnostic value for the development of preeclampsia, eclampsia, HELLP or IUGR, or not. In an embodiment said measuring is done using an immunological assay. In a further aspect, the invention provides a device to determine the predisposition of a pregnant subject to develop preeclampsia, eclampsia, HELLP or IUGR, or not, said device comprising: (a) an analyzing unit comprising a detection agent for ESM-1, which allows the determination of the amount of ESM-1; (b) an evaluation unit comprising a data processor having implemented necessary algorithms for comparing the amount ESM-1 determined with reference values stored in a database in order to determine the predisposition of a pregnant woman to develop preeclampsia, eclampsia, HELLP or IUGR, or not.

In an embodiment, the invention further provides a device to determine the predisposition of a pregnant subject to develop preeclampsia, eclampsia, HELLP or IUGR, or not, said device comprising: (a) an analyzing unit comprising a detection agent for ESM-1, sFlt1, VEGF, P1GF, HGF, sEndoglin, pp-13, PAPP-A or GDF-15, which allows the determination of the amount of ESM-1, sFlt1, VEGF, P1GF, HGF, sEndoglin, pp-13, PAPP-A or GDF-15; (and/or) (b) an evaluation unit comprising a data processor having implemented necessary algorithms for comparing the amount ESM-1, sFlt1, VEGF, P1GF, HGF, sEndoglin, pp-13, PAPP-A or GDF-15 determined with reference values stored in a database and calculate the ratio between the measured markers in order to determine the predisposition of a pregnant woman to develop preeclampsia, eclampsia, HELLP or IUGR, or not. In an embodiment, the invention further provides a device, wherein said evaluation unit is also capable of giving therapeutic recommendations. In yet a further aspect, the invention also provides a kit to determine the predisposition of a pregnant subject to develop preeclampsia, eclampsia, HELLP or IUGR, or not, said kit comprising, at least the detection agent for ESM-1 and preferably, standards which reflect the reference amounts as derived from a pregnant subject or a group thereof known not to suffer or having predisposition to develop preeclampsia, eclampsia, HELLP or IUGR.

The amount of ESM-1 in the biological (fluid) sample of the pregnant subject is especially compared with a reference value derived from a biological (fluid) sample of pregnant subjects having a healthy pregnancy, and wherein the biological samples (i.e. reference samples and the biological sample of the pregnant subject) have been produced in the same way (same type of sample) and in the same week.

The term “substantially” herein, such in “substantially consists”, will be understood by the person skilled in the art. The term “substantially” may also include embodiments with “entirely”, “completely”, “all”, etc. Hence, in embodiments the adjective substantially may also be removed. Where applicable, the term “substantially” may also relate to 90% or higher, such as 95% or higher, especially 99% or higher, even more especially 99.5% or higher, including 100%. The term “comprise” includes also embodiments wherein the term “comprises” means “consists of”. The term “and/or” especially relates to one or more of the items mentioned before and after “and/or”. For instance, a phrase “item 1 and/or item 2” and similar phrases may relate to one or more of item 1 and item 2. The term “comprising” may in an embodiment refer to “consisting of” but may in another embodiment also refer to “containing at least the defined species and optionally one or more other species”. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. However, the terms first and second, etc., may also indicate a relation in time. For instance, a first sample may be extracted earlier in time than a second sample. Especially, this applies to the terms “first occasion” and “second occasion”. Based on the measured values, a diagnosis may be made.

The devices herein may amongst others described during operation. As will be clear to the person skilled in the art, the invention is not limited to methods of operation or devices in operation. It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “to comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage. The invention further applies to a device comprising one or more of the characterizing features described in the description and/or shown in the attached drawings. The invention further pertains to a method or process comprising one or more of the characterising features described in the description and/or shown in the attached drawings.

The various aspects discussed in this patent can be combined in order to provide additional advantages. Furthermore, some of the features can form the basis for one or more divisional applications.

### DESCRIPTION OF THE DRAWINGS

FIG. 1: ESM-1 concentration in plasma during the last trimester of healthy pregnant (P) women (n=32), severe early-onset preeclampsia (SE) women (n=52), and severe late-onset (SL) women (n=8) preeclampsia and from non pregnant (NP) women (n=25). Significance calculate by Mann Whitney test: ****=p<0.0001, ***=p<0.001, and NS=not significant; and FIG. 2. ESM-1 concentrations in plasma during the pregnancy (week 12 until birth) of healthy pregnant (CON; n=23), severe early-onset preeclampsia (SE; n=11) and severe late-onset preeclampsia (SL; n=7). Significance between healthy and preeclamptic pregnancies is calculated by Mann Whitney test: *=p<0.05. The gestation time is indicated with “ge” in weeks (w).

### DETAILED DESCRIPTION OF THE EMBODIMENTS

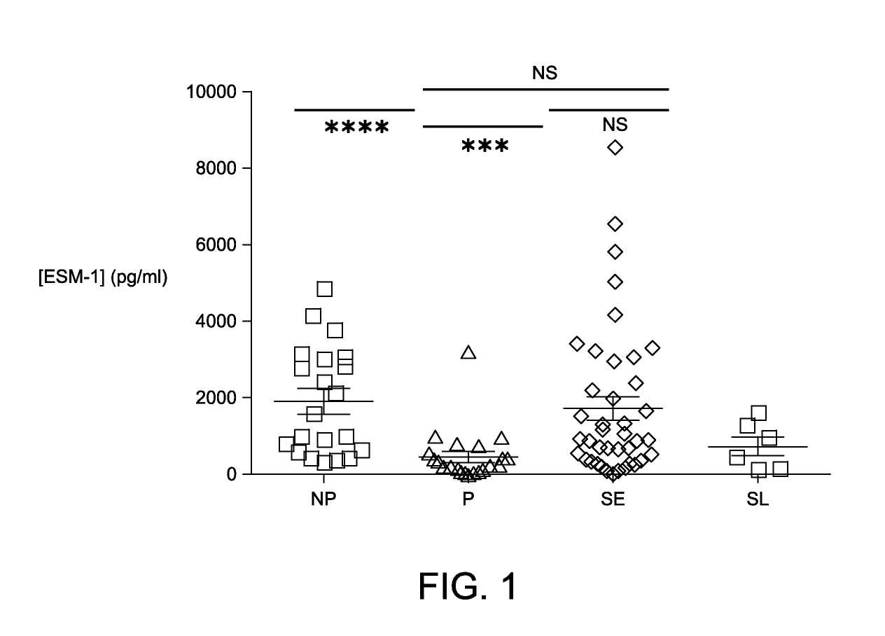

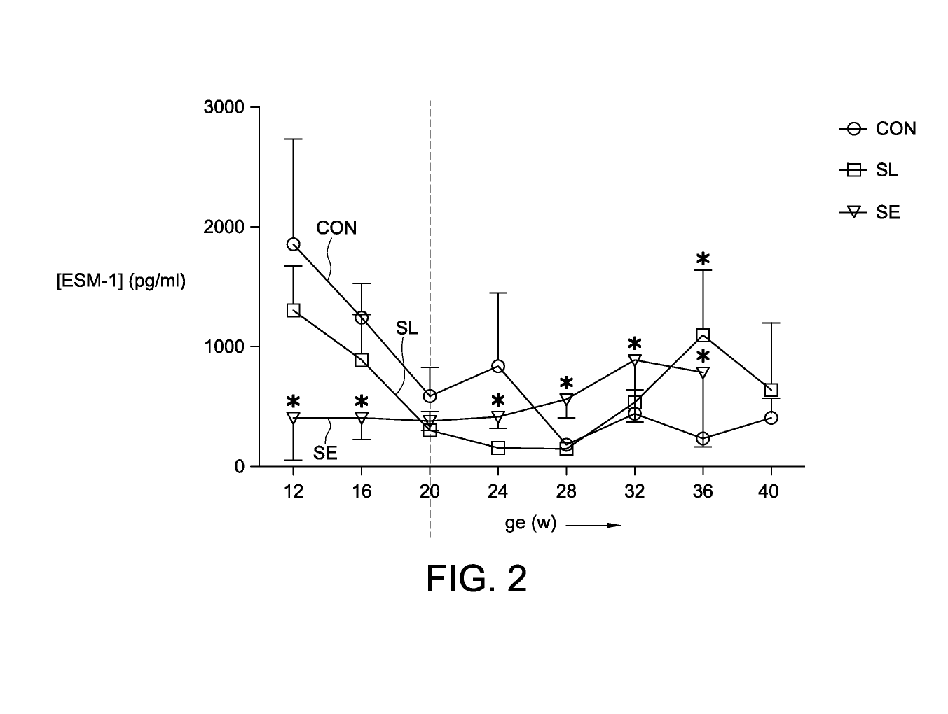

Since ESM-1 plays a role in the regulation of angiogenesis we hypothesized that the expression of ESM-1 during pregnancy would be different between the healthy and the two forms, early and late, of preeclampsia pregnancies. A total of 290 EDTA plasma samples from 23 healthy and GA matched control (CON), 11 severe early-onset PE (SE) and 7 severe late onset PE (SL) pregnancies was collected at regular intervals between week 8 and birth. In these plasma samples ESM-1 was measured by ELISA. For reasons of convenience the mean of the values was determined of 4 week periods; 9 through 12 are expressed as week 12, weeks 13 through 16 are expressed as week 16, etc. During the period between GA week 24 and birth ESM-1 concentrations were significantly increased in both early and late preeclampsia when compared to controls during the same period. Surprisingly the concentration of ESM-1 also differed between the three groups between weeks 12 and 16. The ESM-1 concentration of the healthy pregnancies (mean of 1857 pg/ml) and those that develop severe late-onset PE (mean of 1298 pg/ml) are comparable, but in those pregnancies that develop severe early-onset PE the concentration (mean of 410 pg/ml) is significantly lower (FIG. 1). These results indicate that ESM-1 is a prognostic marker which can determine the risk of women to develop severe early-onset PE already as early as 12 to 16 weeks of gestation (FIG. 2).

Patients with early-onset preeclampsia and healthy pregnant controls were recruited from the antenatal ward and the non-pregnant women were recruited from hospital staff and students. Exclusion criteria for all groups were pre-existing hypertension, diabetes mellitus, vasculitis, renal disease, autoimmune disease, malignancies or women who had recent trauma or surgery. Preeclampsia was defined according to the standards of the International Society for the Study of Hypertension in Pregnancy (ISSHP): diastolic blood pressure of 90 mmHg or more on two or more consecutive occasions more than 4 hrs. apart and proteinuria of more than 300 mg/24 hours. Early-onset preeclamptic women have been included, defined as the onset of preeclampsia before 34 weeks. Blood samples were taken from non-pregnant women and from patients with preeclampsia and healthy pregnant women between week 12 of pregnancy and birth.

Maternal blood samples of both pregnant and preeclamptic women were collected during routine blood sampling. Blood samples were drawn into 10 mL tubes containing EDTA (Venoject, Terumo Europe NV, Leuven, Belgium). From these samples the plasma was collected and frozen (−80° C.) in aliquots until the assays could be performed simultaneously. The plasma levels of all analytes were determined using commercially available reagents. These reagents were either present in sets or kits to perform sandwich ELISA assays. The sFlt, sEng, P1GF and HGF were measured by Quantikine ELISAs (R&D Systems Inc., Minneapolis, US). The ESM-1 was measured using the antibody combination and standard from Lunginnov (Lille, France).

In short; a first mono- or polyclonal antibody specific for the analyte was immobilized in an ELISA micro plate. After overnight incubation the plate was washed and blocked with phosphate buffer saline (PBS) containing 0.1% bovine serum albumin. After subsequent incubation the plate was washed with washing buffer containing 0.1% Tween-20. From a stock solution containing a known concentration of the analyte a standard curve is prepared by 1 to 1 dilution. 100 μL aliquots of the standard curve or the plasma sample were incubated in the different wells of the ELISA plate. After incubation the plate has been washed again and followed by the incubation with a second mono- or polyclonal antibody specific for the analyte and conjugated with biotin. Before incubating the assay with streptavidin-poly-HRPO the plate was washed again. After the incubation with streptavidin-poly-HRPO the assay is in incubated with 3,3′,5,5′-Tetramethylbenzidine (TMB) Substrate solution. The HRPO enzyme present in the well will turn the colorless solution blue proportion to the analyte present. The color development was stopped and the intensity of the color was measured. Further details can be found in the respective package inserts. As indicated above, a total of 290 EDTA plasma samples from 23 healthy and GA matched control (CON), 11 severe early-onset PE (SE) and 7 severe late-onset PE (SL) pregnancies was collected at regular intervals between week 8 and birth. In these plasma samples ESM-1 was measured by ELISA. During the period between GA week 24 and birth ESM-1 concentrations were significantly increased in both early and late preeclampsia when compared to controls during the same period. Surprisingly, the concentration of ESM-1 also differed between the three groups between weeks 12 and 16. The ESM-1 concentration of the healthy pregnancies (mean of 1857 pg/ml) and those that develop severe late-onset PE (mean of 1298 pg/ml) are comparable, but in those pregnancies that develop severe early-onset PE the concentration (mean of 410 pg/ml) is significantly lower (FIG. 1). These results indicate that ESM-1 is a prognostic marker which can determine the risk of women to develop severe early-onset PE already as early as 12 to 16 weeks of gestation (FIG. 2). Based on the measurement of the ESM-1 concentration in blood the risk of pregnant woman to develop preeclampsia later in her pregnancy can be determined. This risk is higher when the ESM-1 concentration is lower than that of women that will not develop preeclampsia or late-onset preeclampsia.

In an aspect, the invention provides a method for identifying a pregnant subject that is likely to have or develop preeclampsia, eclampsia, Hemolysis Elevated Liver enzymes and Low Platelets (HELLP) and/or Intra Uterine Growth Restriction (IUGR), comprising the steps of: (a) extracting a biological sample from a subject; (b) using an assay specific to ESM-1, measuring the amount of ESM-1 in the biological sample; and (c) comparing said amount of ESM-1 to a reference value; and (d) identifying the subject as being likely to have or develop preeclampsia, eclampsia, HELLP and/or IUGR based on a comparison of the amount of ESM-1 to a reference value.

In yet a further aspect, the invention provides a method for identifying a pregnant subject that is likely to have or develop preeclampsia, eclampsia, Hemolysis Elevated Liver enzymes and Low Platelets (HELLP) and/or Intra Uterine Growth Restriction (IUGR), comprising the steps of: (a) extracting a biological sample from a subject; (b) using an assay specific to ESM-1, measuring the amount of ESM-1 in the biological sample; and (c) comparing said amount of ESM-1 to a reference value; and (d) identifying the subject as being likely to have or develop preeclampsia, eclampsia, HELLP and/or IUGR when said amount of ESM-1 is greater than the reference value.

In yet a further aspect, the invention provides a method for identifying a pregnant subject that is likely to have or develop preeclampsia, eclampsia, HELLP and/or IUGR, comprising the steps of: (a) extracting a biological sample from a subject; (b) using an assay specific to ESM-1, measuring the amount of ESM-1 in the biological sample; (c) comparing said amount of ESM-1 to an ESM-1 reference value; and (d) selecting one or more secondary markers from the group consisting of soluble Fms-like tyrosine kinase-1 (sFlt1), Vascular Endothelial Growth Factor (VEGF), Placental Growth Factor (P1GF), Hepatocyte Growth Factor (HGF), soluble Endoglin, placental protein 13 (pp-13), Pregnancy-associated Plasma Protein A (PAPP-A) and Growth Differentiation Factor 15 (GDF-15); (e) using assays specific for each of the one or more secondary markers, measuring the amounts of each of the one or more secondary markers in the biological sample; (f) for each of said one or secondary markers, selecting a secondary marker reference value, and comparing said amount of said secondary marker to the secondary marker reference value; and (g) identifying the subject as being likely to have or develop preeclampsia, eclampsia, HELLP and/or IUGR based on a comparison of the amount ESM-1 with a reference value, and based on a comparison of the amount of said one or more secondary markers with the corresponding secondary marker reference value.

In yet a further aspect, the invention provides a method for identifying a pregnant subject that is likely to have or develop preeclampsia, eclampsia, HELLP and/or IUGR, comprising the steps of: (a) extracting a biological sample from a subject; (b) using an assay specific to ESM-1, measuring the amount of ESM-1 in the biological sample; (c) comparing said amount of ESM-1 to an ESM-1 reference value; and (d) selecting one or more secondary markers from the group consisting of soluble Fms-like tyrosine kinase-1 (sFlt1), Vascular Endothelial Growth Factor (VEGF), Placental Growth Factor (P1GF), Hepatocyte Growth Factor (HGF), soluble Endoglin, placental protein 13 (pp-13), Pregnancy-associated Plasma Protein A (PAPP-A) and Growth Differentiation Factor 15 (GDF-15); (e) using assays specific for each of the one or more secondary markers, measuring the amounts of each of the one or more secondary markers in the biological sample; (f) for each of said one or secondary markers, selecting a secondary marker reference value, and comparing said amount of said secondary marker to the secondary marker reference value; and (g) identifying the subject as being likely to have or develop preeclampsia, eclampsia, HELLP and/or IUGR when said amount of ESM-1 is greater than the ESM-1 reference value, and when said amount of each of said one or more secondary markers is greater than its corresponding secondary marker reference value.

In yet a further aspect, the invention provides a method for identifying a pregnant subject that is likely to have or develop preeclampsia, eclampsia, HELLP and/or IUGR, comprising the steps of: (a) extracting a biological sample from a subject; (b) using an assay specific to ESM-1, measuring the amount of ESM-1 in the biological sample; (c) selecting one or more secondary markers from the group consisting of sFlt1, VEGF, P1GF, HGF, sEndoglin, pp-13, PAPP-A and GDF-15; (d) using assays specific for each of the one or more secondary markers, measuring the amount of each of the one or more secondary markers in the biological sample; (e) for each of the secondary markers: (i) determining the ratio of the amount of ESM-1 to the amount of the secondary marker; and, (ii) selecting a ratio reference value; (iii) comparing the ratio with the ratio reference value; and (f) identifying the subject as being likely to have or develop preeclampsia, eclampsia, HELLP and/or IUGR, especially based on a comparison with the ratio of a reference, such as when for each of the secondary markers, said ratio is greater than the ratio reference value.

In yet a further aspect, the invention provides a method for identifying a pregnant subject that is likely to have or develop preeclampsia, eclampsia, HELLP and/or IUGR, comprising the steps of: (a) extracting a first biological sample from a subject on a first occasion; (b) extracting a second biological sample from a subject on a second occasion; (c) using an assay specific to ESM-1, measuring the amount of ESM-1 in the first biological sample, constituting a first amount; (d) using an assay specific to ESM-1, measuring the amount of ESM-1 in the second biological sample, constituting a second amount; and (e) identifying the subject as being likely to have or develop preeclampsia, eclampsia, HELLP and/or IUGR especially based on a comparison of the first amount and the second amount, such as when the second amount is greater than the first amount by a pre-defined reference value. In yet a further aspect, the invention provides a method for identifying a pregnant subject that is likely to have or develop preeclampsia, eclampsia, HELLP and/or IUGR, comprising the steps of (a) extracting a first biological sample from a subject; (b) extracting a second biological sample from a subject; (c) using an assay specific to ESM-1, measuring the amount of ESM-1 in the first biological sample, constituting a first amount of ESM-1; (d) using an assay specific to ESM-1, measuring the amount of ESM-1 in the second biological sample, constituting a second amount of ESM-1; (e) selecting one or more secondary markers from the group consisting of sFlt1, VEGF, P1GF, HGF, sEndoglin, pp-13, PAPP-A and GDF-15; (f) using assays specific for each of the one or more secondary markers, measuring the amounts of each of the one or more secondary markers in the first biological sample, constituting a set of first amounts of the secondary markers; (g) using assays specific for each of the one or more secondary markers, measuring the amounts of each of the one or more secondary markers in the second biological sample, constituting a set of second amounts of the secondary markers; (h) identifying the subject as being likely to have or develop preeclampsia, eclampsia, HELLP and/or IUGR especially based (i) on a comparison of the first amount of ESM-1 and the second amount of ESM-1 and/or based (ii) on a comparison of the second amount of the secondary marker and the first amount, such as when the second amount of ESM-1 is greater than the first amount of ESM-1 by a pre-defined ESM-1 reference value, and for each of the secondary markers, the second amount of the secondary marker is greater than the first amount of the secondary marker by a pre-defined reference value for that secondary marker.

In yet a further aspect, the invention provides a method for identifying a pregnant subject that is likely to have or develop preeclampsia, eclampsia, HELLP and/or IUGR, comprising the steps of (a) extracting a first biological sample from a subject; (b) extracting a second biological sample from a subject; (c) using an assay specific to ESM-1, measuring the amount of ESM-1 in the first biological sample, constituting a first amount of ESM-1; (d) using an assay specific to ESM-1, measuring the amount of ESM-1 in the second biological sample, constituting a second amount of ESM-1; (e) selecting one or more secondary markers from the group consisting of sFlt1, VEGF, P1GF, HGF, sEndoglin, pp-13, PAPP-A and GDF-15; (f) using assays specific for each of the one or more secondary markers, measuring the amounts of each of the one or more secondary markers in the first biological sample, constituting a set of first amounts of the secondary markers; (g) using assays specific for each of the one or more secondary markers, measuring the amounts of each of the one or more secondary markers in the second biological sample, constituting a set of second amounts of the secondary markers; (h) for each of the secondary markers: (1) determining the ratio of the first amount of ESM-1 to the first amount of the secondary marker, constituting a first ratio; (2) determining the ratio of the second amount of ESM-1 to the second amount of the secondary marker, constituting a second ratio; (3) comparing the first ratio to the second ratio; and (i) identifying the subject as being likely to have or develop preeclampsia, eclampsia, HELLP and/or IUGR, especially when for each of the secondary markers, the second ratio is greater than the first ratio by a pre-defined reference value.

In an embodiment, he method further comprises the step of uterine artery Doppler screening. In an embodiment, said step(s) of measuring are done using one or more immunological assays, especially wherein the one or more immunological assay comprise an ELISA (Enzyme-linked immunosorbent assay) assay, a turbidimetric assay, radioimmunosorbent assay (RIST), radioimmunoassay (RIA), direct or indirect immunofluorescence, particle gel immuno assay (PaGIA), mass spectrometry (MS) or lateral flow assay. In an embodiment, said immunological assay(s) are an ELISA. In an embodiment, said immunological assay(s) are a lateral flow assay. In an embodiment, said step(s) of measuring are done using one or more enzymatic colorimetric assays. In an embodiment, said step(s) of measuring are done using mass spectrometry (MS). In an embodiment, said amount of ESM-1 is the level of free ESM-1, bound ESM-1, a metabolite of ESM-1 or total ESM-1.

In yet a further aspect, the invention provides a device for identifying a pregnant subject that is likely to have or develop preeclampsia, eclampsia, HELLP and/or IUGR, said device comprising: (a) An analyzing unit comprising a detection agent for ESM-1, for instance being an antibody, a (recombinant) receptor for ESM-1 or an aptamer, which allows the determination of the amount of ESM-1; (b) An evaluation unit comprising a data processor having implemented necessary algorithms for comparing the amount ESM-1 determined with reference values stored in a database in order to determine whether a pregnant subject is likely to have or develop preeclampsia, eclampsia, HELLP and/or IUGR. In yet a further aspect, the invention provides a device for identifying a pregnant subject that is likely to have or develop preeclampsia, eclampsia, HELLP and/or IUGR, said device comprising: (a) An analyzing unit comprising a detection agent for ESM-1, sFlt1, VEGF, P1GF, HGF, sEndoglin, pp-13, PAPP-A or GDF-15, which allows the determination of the amount of ESM-1, sFlt1, VEGF, P1GF, HGF, sEndoglin, pp-13, PAPP-A or GDF-15; (b) An evaluation unit comprising a data processor having implemented necessary algorithms for comparing the amount ESM-1, sFlt1, VEGF, P1GF, HGF, sEndoglin, pp-13, PAPP-A or GDF-15 determined with reference values stored in a database and calculate the ratio between the measured markers in order to determine whether a pregnant subject is likely to have or develop preeclampsia, eclampsia, HELLP and/or IUGR. In an embodiment, said evaluation unit is also capable of giving therapeutic recommendations. In yet a further aspect, the invention provides a use of an evaluation of the amount of ESM-1 in an extracted biological sample from a pregnant subject for identifying whether the pregnant subject is likely to have or develop preeclampsia, eclampsia, HELLP and/or IUGR.

In yet a further aspect, the invention provides a in vitro use of an extracted biological sample from a pregnant subject for identifying whether the pregnant subject is likely to have or develop preeclampsia, eclampsia, HELLP and/or IUGR, the use comprising: (a) using an assay specific to ESM-1, measuring the amount of ESM-1 in the biological sample; (b) comparing said amount of ESM-1 to a reference value; and (c) identifying the subject as being likely to have or develop preeclampsia, eclampsia, HELLP and/or IUGR. In an embodiment, the use (further) comprises identifying the subject as being likely to have or develop preeclampsia, eclampsia, HELLP and/or IUGR based on a comparison of the amount of ESM-1 to a reference value. In an embodiment, the use (further) comprises identifying the subject as being likely to have or develop preeclampsia, eclampsia, HELLP and/or IUGR when said amount of ESM-1 is greater than the reference value, especially when the biological sample is from a pregnant subject in any one week of gestation between week 26 or later of gestation.

In yet a further aspect, the invention provides a in vitro use of an extracted biological sample from a pregnant subject for identifying whether the pregnant subject is likely to have or develop preeclampsia, eclampsia, HELLP and/or IUGR, the use comprising: (a) using an assay specific to ESM-1, measuring the amount of ESM-1 in the biological sample; (b) comparing said amount of ESM-1 to an ESM-1 reference value; and (c) selecting one or more secondary markers from the group consisting of sFlt1, VEGF, P1GF, HGF, sEndoglin, pp-13, PAPP-A and GDF-15; (d) using assays specific for each of the one or more secondary markers, measuring the amounts of each of the one or more secondary markers in the biological sample; (e) for each of said one or secondary markers, selecting a secondary marker reference value, and comparing said amount of said secondary marker to the secondary marker reference value; and (f) identifying the subject as being likely to have or develop preeclampsia, eclampsia, HELLP and/or IUGR.

In an embodiment, the use (further) comprises identifying the subject as being likely to have or develop preeclampsia, eclampsia, HELLP and/or IUGR based on a comparison of the amount ESM-1 with a reference value, and based on a comparison of the amount of said one or more secondary markers with the corresponding secondary marker reference value. In an embodiment, the use (further) comprises identifying the subject as being likely to have or develop preeclampsia, eclampsia, HELLP and/or IUGR when said amount of ESM-1 is greater than the ESM-1 reference value, especially when the biological sample is from a pregnant subject in any one week of gestation between week 26 or later of gestation. In an embodiment, the use (further) comprises identifying the subject as being likely to have or develop preeclampsia, eclampsia, HE LLP and/or IUGR when said amount of each of said one or more secondary markers is greater than its corresponding secondary marker reference value.

In yet a further aspect, the invention provides a in vitro use of an extracted biological sample from a pregnant subject for identifying a pregnant subject that is likely to have or develop preeclampsia, eclampsia, HELLP and/or IUGR, comprising the steps of: (a) using an assay specific to ESM-1, measuring the amount of ESM-1 in the biological sample; (b) selecting one or more secondary markers from the group consisting of sFlt1, VEGF, P1GF, HGF, sEndoglin, pp-13, PAPP-A and GDF-15; (c) using assays specific for each of the one or more secondary markers, measuring the amount of each of the one or more secondary markers in the biological sample; (d) for each of the secondary markers: (i) determining the ratio of the amount of ESM-1 to the amount of the secondary marker; and, (ii) selecting a ratio reference value; (iii) comparing the ratio with the ratio reference value; and (e) identifying the subject as being likely to have or develop preeclampsia, eclampsia, HELLP and/or IUGR.

In an embodiment, the use (further) comprises identifying the subject as being likely to have or develop preeclampsia, eclampsia, HELLP and/or IUGR based on a comparison for each of the secondary markers with the ratio reference value. In an embodiment, the use (further) comprises identifying the subject as being likely to have or develop preeclampsia, eclampsia, HELLP and/or IUGR when for each of the secondary markers, said ratio is greater than the ratio reference value. In an embodiment said sample is derived from a subject being in any one week of gestation between week 1 to 20 of gestation, especially between weeks 12 to 16, and identifying the subject as being likely to have or develop preeclampsia, eclampsia, HELLP and/or IUGR when said amount of ESM-1 is smaller than the ESM-1 reference value.

In yet a further aspect, the invention provides a in vitro use of extracted biological samples from a pregnant subject for identifying a pregnant subject that is likely to have or develop preeclampsia, eclampsia, HELLP and/or IUGR, using a first biological sample from a subject extracted on a first occasion and using a second biological sample from a subject extracted on a second occasion, comprising the steps of: (a) using an assay specific to ESM-1, measuring the amount of ESM-1 in the first biological sample, constituting a first amount; (b) using an assay specific to ESM-1, measuring the amount of ESM-1 in the second biological sample, constituting a second amount; and (c) identifying the subject as being likely to have or develop preeclampsia, eclampsia, HELLP and/or IUGR. In an embodiment, the use (further) comprises identifying the subject as being likely to have or develop preeclampsia, eclampsia, HELLP and/or IUGR based on a comparison of the second amount and the first amount. In an embodiment, the use (further) comprises identifying the subject as being likely to have or develop preeclampsia, eclampsia, HELLP and/or IUGR when the second amount is greater than the first amount by a pre-defined reference value, especially when the biological sample is from a pregnant subject in any one week of gestation between week 26 or later of gestation. In an embodiment, the use (further) comprises identifying the subject as being likely to have or develop preeclampsia, eclampsia, HELLP and/or IUGR when the second amount is smaller than the first amount by a pre-defined reference value when the biological sample is from a pregnant subject in any one week of gestation before week 26, especially before week 20, even more especially in any week of gestation between week 12 and 16 of gestation.

In yet a further aspect, the invention provides a in vitro use of extracted biological samples from a pregnant subject for identifying a pregnant subject that is likely to have or develop preeclampsia, eclampsia, HELLP and/or IUGR, using a first biological sample from a subject extracted on a first occasion and using a second biological sample from a subject extracted on a second occasion, comprising the steps of: (a) using an assay specific to ESM-1, measuring the amount of ESM-1 in the first biological sample, constituting a first amount of ESM-1; (b) using an assay specific to ESM-1, measuring the amount of ESM-1 in the second biological sample, constituting a second amount of ESM-1; (c) selecting one or more secondary markers from the group consisting of sFlt1, VEGF, P1GF, HGF, sEndoglin, pp-13, PAPP-A and GDF-15; (d) using assays specific for each of the one or more secondary markers, measuring the amounts of each of the one or more secondary markers in the first biological sample, constituting a set of first amounts of the secondary markers; (e) using assays specific for each of the one or more secondary markers, measuring the amounts of each of the one or more secondary markers in the second biological sample, constituting a set of second amounts of the secondary markers; (0 identifying the subject as being likely to have or develop preeclampsia, eclampsia, HELLP and/or IUGR.

In an embodiment, the use (further) comprises identifying the subject as being likely to have or develop preeclampsia, eclampsia, HELLP and/or IUGR when the second amount is greater than the first amount by a pre-defined reference value, especially when the biological sample is from a pregnant subject in any one week of gestation between week 26 or later of gestation. In an embodiment, the use (further) comprises identifying the subject as being likely to have or develop preeclampsia, eclampsia, HELLP and/or IUGR when the second amount is smaller than the first amount by a pre-defined reference value when the biological sample is from a pregnant subject in any one week of gestation before week 26, especially before week 20, even more especially in any week of gestation between week 12 and 16 of gestation.

In an embodiment, the use (further) comprises identifying the subject as being likely to have or develop preeclampsia, eclampsia, HELLP and/or IUGR based on a comparison of each of said one or more secondary markers with its corresponding secondary marker reference value. In an embodiment, the use (further) comprises identifying the subject as being likely to have or develop preeclampsia, eclampsia, HELLP and/or IUGR when said amount of each of said one or more secondary markers is (also) greater than its corresponding secondary marker reference value.

In yet a further aspect, the invention provides a in vitro use of extracted biological samples from a pregnant subject for identifying a pregnant subject that is likely to have or develop preeclampsia, eclampsia, HELLP and/or IUGR, using a first biological sample from a subject extracted on a first occasion and using a second biological sample from a subject extracted on a second occasion, comprising the steps of: (a) using an assay specific to ESM-1, measuring the amount of ESM-1 in the first biological sample, constituting a first amount of ESM-1; (b) using an assay specific to ESM-1, measuring the amount of ESM-1 in the second biological sample, constituting a second amount of ESM-1; (c) selecting one or more secondary markers from the group consisting of sFlt1, VEGF, P1GF, HGF, sEndoglin, pp-13, PAPP-A and GDF-15; (d) using assays specific for each of the one or more secondary markers, measuring the amounts of each of the one or more secondary markers in the first biological sample, constituting a set of first amounts of the secondary markers; (e) using assays specific for each of the one or more secondary markers, measuring the amounts of each of the one or more secondary markers in the second biological sample, constituting a set of second amounts of the secondary markers; (f) for each of the secondary markers: (i) determining the ratio of the first amount of ESM-1 to the first amount of the secondary marker, constituting a first ratio; (ii) determining the ratio of the second amount of ESM-1 to the second amount of the secondary marker, constituting a second ratio; (iii) comparing the first ratio to the second ratio; and (g) identifying the subject as being likely to have or develop preeclampsia, eclampsia, HELLP and/or IUGR.

In an embodiment, the use (further) comprises identifying the subject as being likely to have or develop preeclampsia, eclampsia, HELLP and/or IUGR based on a comparison of the ratio each of the secondary markers with a pre-defined reference value. In an embodiment, the use (further) comprises identifying the subject as being likely to have or develop preeclampsia, eclampsia, HELLP and/or IUGR when for each of the secondary markers, the second ratio is greater than the first ratio by a pre-defined reference value. In an embodiment, said step(s) of measuring are done using one or more immunological assays. In an embodiment, said immunological assay(s) are an ELISA. In an embodiment, said immunological assay(s) are a lateral flow assay. In an embodiment, said step(s) of measuring are done using one or more of an enzymatic or a colorimetric or a luminescent assay. In an embodiment, said step(s) of measuring are done using mass spectrometry (MS). In an embodiment, said amount of ESM-1 is the level of free ESM-1, bound ESM-1, a metabolite of ESM-1 or total ESM-1.

In an embodiment, one or more of said first biological sample and said second biological sample, especially both the first biological sample and the second biological sample, is a blood, plasma, serum, urine or saliva from the subject. In an embodiment, one or more of said first biological sample and said second biological sample, especially both the first biological sample and the second biological sample, is derived from a subject being in any one week of gestation between week 1 to 20 of gestation, especially between weeks 12 to 16. In an embodiment, one or more of said first biological sample and said second biological sample, especially both the first biological sample and the second biological sample, is derived from a subject being in any one week of gestation between week 20 to 36 of gestation.

In yet a further aspect, the invention provides a kit for identifying a pregnant subject that is likely to have or develop preeclampsia, eclampsia, HELLP and/or IUGR, said kit comprising: (a) An analyzing unit comprising a detection agent for ESM-1; (b) an evaluation unit for determining whether a pregnant subject is likely to have or develop preeclampsia, eclampsia, HELLP and/or IUGR, based on a result provided by the analyzing unit. In an embodiment, the kit may further comprise a manual or a reference to a (remote) manual. In an embodiment, the manual includes instructions how to extract biological sample from a pregnant subject and/or how to use the analyzing unit and/or how to use the evaluation unit. In an embodiment, the evaluation unit comprises a color scheme, wherein the analyzing unit is configured to provide a color reaction wherein the color is dependent upon the amount of ESM-1 in an extracted biological sample from a pregnant subject. In an embodiment, the kit (further) comprises: (a) An evaluation unit comprising a data processor for determining whether a pregnant subject is likely to have or develop preeclampsia, eclampsia, HELLP and/or IUGR. In an embodiment, the kit (further) comprises: (b) An evaluation unit comprising a data processor having implemented necessary algorithms for comparing the amount ESM-1 determined with reference values stored in a database in order to determine whether a pregnant subject is likely to have or develop preeclampsia, eclampsia, HELLP and/or IUGR.

In yet a further aspect, the invention provides a kit for identifying a pregnant subject that is likely to have or develop preeclampsia, eclampsia, HELLP and/or IUGR, said kit comprising: (a) An analyzing unit comprising a detection agent for ESM-1, sFlt1, VEGF, P1GF, HGF, sEndoglin, pp-13, PAPP-A or GDF-15. In yet a further aspect, the invention provides a kit as described herein for identifying a pregnant subject that is likely to have or develop preeclampsia, eclampsia, HELLP and/or IUGR, said kit comprising: (a) An analyzing unit comprising a detection agent for ESM-1, sFlt1, VEGF, P1GF, HGF, sEndoglin, pp-13, PAPP-A or GDF-15, which allows the determination of the amount of ESM-1, sFlt1, VEGF, P1GF, HGF, sEndoglin, pp-13, PAPP-A or GDF-15.

In yet a further aspect, the invention provides a kit as described herein, said kit (further) comprising: (b) An evaluation unit comprising a data processor having implemented necessary algorithms for comparing the amount ESM-1, sFlt1, VEGF, P1GF, HGF, sEndoglin, pp-13, PAPP-A or GDF-15 determined with reference values stored in a database and calculate the ratio between the measured markers in order to determine whether a pregnant subject is likely to have or develop preeclampsia, eclampsia, HELLP and/or IUGR. In an embodiment, said evaluation unit is also capable of giving therapeutic recommendations. In an embodiment, the analyzing unit comprises an assay. In an embodiment, the analyzing unit comprises an ELISA (Enzyme-linked immunosorbent assay) assay, a turbidimetric assay, radioimmunosorbent assay (RIST), radioimmunoassay (RIA), direct or indirect immunofluorescence, particle gel immuno assay (PaGIA), mass spectrometry (MS) or lateral flow assay. In an embodiment, the manual includes information to extract biological sample from a pregnant subject in any one week of gestation, especially between week 1 to 20 of gestation, even more especially between weeks 12 to 16. The method and use, as well as the application of the kit may include taking a plurality of samples over a period of time and determining with the ESM-1 specific assay the amount(s) of ESM-1 or derivative values thereof like ratio's of values. Alternative or additionally, the quantification is an indirect quantification, for instance by a color reaction that may be dependent upon the concentration (amount). Based on the color, a prediction may be made about a pregnant subject whether she is likely to have or develop preeclampsia, eclampsia, Hemolysis Elevated Liver enzymes and Low Platelets (HELLP) and/or Intra Uterine Growth Restriction (IUGR).

Additionally or alternatively, reference values may be determined or provided, for instance in a manual (on the internet) based upon one can determine whether the pregnant subject has or is likely to develop one of the above-mentioned syndromes. When comparing the value obtained of the subject with a reference value, or a plurality of reference values when more than one parameter is determined, the status of likely status to be can be predicted. Hence, the manual and/or reference data can be remote, such as on the internet. The user may derive the date from the internet.

Measuring can be done using a method to quantify the amount of protein via the amount of RNA coding for the said protein. Hence, in an embodiment said step(s) of measuring are done using a method to quantify the amount of protein via the amount of RNA coding for the said protein. Further, additionally or alternatively said quantification can be done via RT-PCR, PCR, hybridization or other means to determine the amount of nucleotides.

1-42. (canceled)

43. An in vitro method for identifying from a biological fluid sample from a pregnant subject a pregnancy related syndrome selected from the group consisting of Preeclampsia, Eclampsia, Hemolysis Elevated Liver enzymes and Low Platelets (HELLP), and Intra Uterine Growth Restriction (IUGR), the method including (i) measuring the amount of ESM-1 in the biological sample, wherein the biological sample is from the pregnant subject being in any one week of gestation between week 1 to 20 of gestation; (ii) comparing said amount of ESM-1 to a reference value, and (iii) identifying the subject as being likely to have or develop the pregnancy related syndrome based on a comparison of the amount of ESM-1 to the reference value.

44. The method according to claim 43, further comprising (iii) identifying the subject as being likely to have or develop the pregnancy related syndrome based on a comparison of the amount of ESM-1 to the reference value when said amount of ESM-1 is smaller than the ESM-1 reference value, wherein the reference value is the amount of ESM-1 in biological samples of pregnant subjects having a healthy pregnancy, wherein said biological fluid sample comprises a blood sample, a plasma sample, or a serum sample from said pregnant subject.

45. The method according to claim 43, wherein said biological fluid sample is from a pregnant subject being in any one week of gestation between weeks 12 to 16 of gestation, and wherein said biological fluid sample comprises a plasma sample and wherein the pregnant subject is evaluated to have the pregnancy related syndrome when the ESM-1 value is in the range of 410±355 pg/ml.

46. The method according claim 43, wherein the amount of ESM-1 of the biological fluid sample of the pregnant subject is indicative of the pregnancy related syndrome when the amount is equal to or less than 70% of the amount of ESM-1 of biological fluid samples of pregnant subjects having a healthy pregnancy.

47. The method according to claim 43, further comprising using an assay specific to ESM-1, wherein measuring the amount of ESM-1 in the biological sample includes using mass spectrometry (MS);
using uterine artery Doppler screening and deriving thereof information about the placentation wherein the assay is selected from the group consisting of an ELISA (Enzyme-linked immunosorbent assay) assay, a turbidimetric assay, radioimmunosorbent assay (RIST), a radioimmunoassay (RIA), a direct immunofluorescence assay, an indirect immunofluorescence assay, a particle gel immuno assay (PaGIA), a lateral flow assay an enzymatic assay, a colorimetric assay, and a luminescent assay.

48. The method according to claim 43, wherein said amount of ESM-1 is the level of free ESM-1 in the biological sample.

49. The method according to claim 43, wherein one or more of (a) measuring the amount of ESM-1 in the biological sample comprises a method to do a quantification of RNA coding for ESM-1 in cells or cell-free RNA in the biological sample, and (b) measuring the amount of ESM-1 in the biological sample comprises quantifying the amount of protein via the amount of RNA coding for ESM-1; and wherein said quantification is done via RT-PCR, PCR, hybridization or other means to determine the amount of nucleotides.

50. The method according to claim 43, further comprising (iv) using a secondary marker selected from the group consisting of soluble Fms-like tyrosine kinase-1 (sFlt1), Vascular Endothelial Growth Factor (VEGF), Placental Growth Factor (P1GF), Hepatocyte Growth Factor (HGF), soluble Endoglin, placental protein 13 (pp-13), Pregnancy-associated Plasma Protein A (PAPP-A) Growth Differentiation Factor 15 (GDF-15), pikachurin and a hemopexin, (v) optionally using an assay specific for the secondary marker, and measuring the amount of the secondary marker in the biological sample, and optionally comparing said amount of said secondary marker to a secondary marker reference value, the method further comprising identifying the subject as being likely to have or develop the pregnancy related syndrome based on a comparison of the amount of ESM-1 to a reference value, and based on a comparison of the amount of said one or more secondary markers with the corresponding secondary marker reference value, and the method, further comprising determining a ratio of the amount of ESM-1 to the amount of the secondary marker, selecting a ratio reference value, and comparing the ratio with the ratio reference value, and the method further comprising identifying the subject as being likely to have or develop the pregnancy related syndrome based on a comparison of the amount of ESM-1 to a reference value, and based on a comparison of the ratio of the amount of ESM-1 to the amount of the secondary marker to the ratio reference value.

51. The method according to claim 43, the method comprising providing a first biological sample from a subject extracted on a first occasion, and providing a second biological sample from the subject extracted on a second occasion, using an assay specific to ESM-1, measuring the amount of ESM-1 in the first biological sample, constituting a first amount, using an assay specific to ESM-1, measuring the amount of ESM-1 in the second biological sample, and constituting a second amount, and the method further comprising identifying the subject as being likely to have or develop the pregnancy related syndrome based on a comparison of the first amount and the second amount.

52. A device for identifying a pregnant subject that is likely to have or develop a pregnancy related syndrome selected from the group consisting of Preeclampsia, Eclampsia, HELLP and IUGR, said device comprising (a) an analyzing unit comprising a detection agent for ESM-1, for instance being an antibody, a (recombinant) receptor for ESM-1 or an aptamer, which allows the determination of the amount of ESM-1; (b) an evaluation unit comprising a data processor having implemented necessary algorithms for comparing the amount of ESM-1 determined with a reference value stored in a database in order to determine whether a pregnant subject is likely to have or develop preeclampsia, eclampsia, HELLP and/or IUGR.

53. The device according to claim 52, further comprising an analyzing unit comprising a detection agent for sFlt1, VEGF, P1GF, HGF, sEndoglin, pp-13, PAPP-A or GDF-15, which allows the determination of the amount of sFlt1, VEGF, P1GF, HGF, sEndoglin, pp-13, PAPP-A or GDF-15, and an evaluation unit comprising a data processor having implemented necessary algorithms for comparing the amount of sFlt1, VEGF, P1GF, HGF, sEndoglin, pp-13, PAPP-A or GDF-15 determined with reference values stored in a database and calculate the ratio between the measured markers in order to determine whether a pregnant subject is likely to have or develop preeclampsia, eclampsia, HELLP and/or IUGR, and wherein said evaluation unit is also capable of giving therapeutic recommendations.

54. A method of evaluating a risk of a condition, the method comprising evaluating an amount of ESM-1 in an extracted biological fluid sample from a pregnant subject; and
based on said amount determining whether the pregnant subject is likely to have or develop a pregnancy related syndrome selected from the group consisting of Preeclampsia, Eclampsia, HELLP and IUGR.

55. An in-vitro method of evaluating a risk of a condition, the in-vitro method comprising extracting biological fluid sample from a pregnant subject; and
based on the extracted biological fluid sample, determining whether the pregnant subject is likely to have or develop a pregnancy related syndrome selected from the group consisting of Preeclampsia, Eclampsia, HELLP and IUG, the use comprising: (a) measuring the amount of ESM-1 in the biological sample, especially using an assay specific to ESM-1; (b) comparing said amount of ESM-1 to a reference value; and (c) identifying the subject as being likely to have or develop the pregnancy related syndrome.

56. A kit for identifying whether a pregnant subject is likely to have or develop a pregnancy related syndrome selected from the group consisting of Preeclampsia, Eclampsia, HELLP and IUG, said kit comprising: (a) An analyzing unit comprising a detection agent for ESM-1; (b) An evaluation unit for determining whether a pregnant subject is likely to have or develop the pregnancy related syndrome, based on a result provided by the analyzing unit.

57. The kit according to claim 56, further comprising a manual or a reference to a manual, wherein the manual includes instructions how to extract biological fluid sample from a pregnant subject and/or how to use the analyzing unit and/or how to use the evaluation unit, wherein the manual includes information to extract biological fluid sample from a pregnant subject in any one week of gestation, especially between week 1 to 20 of gestation, even more especially between weeks 12 to 16, and wherein the evaluation unit comprises a color scheme, wherein the analyzing unit is configured to provide a color reaction wherein the color is dependent upon the amount of ESM-1 in an extracted biological sample from a pregnant subject.

58. The kit according to claim 56, comprising (i) An analyzing unit comprising a detection agent for ESM-1 and one or more of sFlt1, VEGF, P1GF, HGF, sEndoglin, pp-13, PAPP-A and GDF-15, especially the analyzing unit comprising a detection agent for ESM-1, and a detection agent for one or more of sFlt1, VEGF, P1GF, HGF, sEndoglin, pp-13, PAPP-A and GDF-15, which allows the determination of the amount of ESM-1, and one or more of sFlt1, VEGF, P1GF, HGF, sEndoglin, pp-13, PAPP-A and GDF-15, wherein one or more of (a) the analyzing unit comprises an assay, especially wherein the analyzing unit comprises an ELISA (Enzyme-linked immunosorbent assay) assay, a turbidimetric assay, radioimmunosorbent assay (RIST), radioimmunoassay (RIA), direct or indirect immunofluorescence, particle gel immuno assay (PaGIA), or a lateral flow assay, and (b) the analyzing unit comprises a method to do a quantification of the RNA coding for ESM-1 in cells or cell-free RNA in the biological fluid sample; and (ii) An evaluation unit comprising a data processor for determining whether a pregnant subject is likely to have or develop preeclampsia, eclampsia, HELLP and/or IUGR, especially a data processor having implemented necessary algorithms for comparing the amount ESM-1 determined with reference values stored in a database in order to determine whether a pregnant subject is likely to have or develop the pregnancy related syndrome.

59. The kit according to claim 56, wherein said evaluation unit comprising a data processor having implemented necessary algorithms for comparing the amount ESM-1, and one or more of sFlt1, VEGF, P1GF, HGF, sEndoglin, pp-13, PAPP-A and GDF-15 determined with reference values stored in a database and calculate the ratio between the measured markers in order to determine whether a pregnant subject is likely to have or develop the pregnancy related syndrome.

60. The kit according to claim 56, further comprising a unit, for predicting a pregnant subject in any one of week 1 to 20 of gestation to develop after week 20 early onset severe preeclampsia, based on a measurement of the amount of ESM-1 in a biological fluid sample from the pregnant subject, wherein the biological fluid sample comprises a blood sample, a plasma sample, or a serum sample from said pregnant subject.

61. The kit according claim 57, comprising (i) an analyzing unit comprising a detection agent for ESM-1 and one or more of sFlt1, VEGF, P1GF, HGF, sEndoglin, pp-13, PAPP-A and GDF-15, especially the analyzing unit comprising a detection agent for ESM-1, and a detection agent for one or more of sFlt1, VEGF, P1GF, HGF, sEndoglin, pp-13, PAPP-A and GDF-15, which allows the determination of the amount of ESM-1, and one or more of sFlt1, VEGF, P1GF, HGF, sEndoglin, pp-13, PAPP-A and GDF-15, wherein one or more of (a) the analyzing unit comprises an assay, especially wherein the analyzing unit comprises an ELISA (Enzyme-linked immunosorbent assay) assay, a turbidimetric assay, radioimmunosorbent assay (RIST), radioimmunoassay (RIA), direct or indirect immunofluorescence, particle gel immuno assay (PaGIA), or a lateral flow assay, and (b) the analyzing unit comprises a method to do a quantification of the RNA coding for ESM-1 in cells or cell-free RNA in the biological fluid sample; and (ii) an evaluation unit comprising a data processor for determining whether a pregnant subject is likely to have or develop preeclampsia, eclampsia, HELLP and/or IUGR, especially a data processor having implemented necessary algorithms for comparing the amount ESM-1 determined with reference values stored in a database in order to determine whether a pregnant subject is likely to have or develop the pregnancy related syndrome.

62. The kit according to claim 57, said kit comprising an evaluation unit comprising a data processor having implemented necessary algorithms for comparing the amount ESM-1, and one or more of sFlt1, VEGF, P1GF, HGF, sEndoglin, pp-13, PAPP-A and GDF-15 determined with reference values stored in a database and calculate the ratio between the measured markers in order to determine whether a pregnant subject is likely to have or develop the pregnancy related syndrome.
